(19) United States
(12) Patent Application Publication
(10) Pub. No.: US 2024/0293088 A1
(43) Pub. Date: Sep. 5, 2024

(54) Systems and Methods for Predictive Glucose Management

(71) Applicant: The Board of Trustees of the Leland Stanford Junior University, Stanford, CA (US)

(72) Inventors: Yuhao Huang, Stanford, CA (US); Casey Halpern, Philadelphia, PA (US); Jonathon Parker, Scottsdale, AZ (US); Rajat Shivacharan, Stanford, CA (US); Jeffrey B. Wang, Stanford, CA (US)

(73) Assignee: The Board of Trustees of the Leland Stanford Junior University, Stanford, CA (US)

(21) Appl. No.: 18/574,287

(22) PCT Filed: Aug. 30, 2022
(86) PCT No.: PCT/US2022/075695

§ 371 Date: Dec. 26, 2023

Related U.S. Application Data
(60) Provisional application No. 63/238,583, filed on Aug. 30, 2021.

(51) Int. Cl. A61B 5/00; A61B 5/145

(52) U.S. Cl. CPC A61B 5/7278; A61B 5/14532; A61B 5/4064; A61B 5/4839; A61B 5/7221; A61B 5/7275; A61B 5/746

(57) ABSTRACT

Systems and methods for predictive glucose in accordance with embodiments of the invention are illustrated. One embodiment includes glucose management device, including a brain signal recorder, and a controller, including a processor, and a memory, the memory containing a glucose monitoring application configured to direct the processor to record a brain activity signal of a user's brain using the brain signal recorder, and decode the brain activity signal to predict future glucose levels of the patient.

### CROSS-REFERENCE TO RELATED APPLICATIONS

The current application is a national stage application of PCT Application No. PCT/US2022/075695 entitled “Systems and Methods for Predictive Glucose Management” filed Aug. 30, 2022, which claims the benefit of and priority under 35 U.S.C. § 119(e) to U.S. Provisional Patent Application No. 63/238,583 entitled “Systems, Methods, and Composition of Decoding Glucose Using Brain Activity” filed Aug. 30, 2021. The disclosures of PCT Patent Application No. PCT/US2022/075695 and U.S. Provisional Patent Application No. 63/238,583 are hereby incorporated by reference in their entirety for all purposes.

### FIELD OF THE INVENTION

The present invention generally relates to the prediction of future glucose levels in a patient based on brain activity, and preemptive management based on said prediction.

### BACKGROUND

Glucose is a simple sugar that is a critical energy source for all known life. Glucose levels are typically split into two different measurements. Blood glucose is a measurement of glucose in blood and is generally measured as the venous plasma level, where it tends to be 3-5 mg/mL less than in arterial blood because some of the glucose diffuses from the plasma to interstitial fluid as blood circulates through the capillary system. Interstitial glucose, in contrast, is a measurement of glucose in the interstitial fluid of the body. Interstitial glucose and blood glucose measurements often substantially differ even if measured at the same time.

Hyperglycemia is a medical condition in which the patient has an excessive amount of glucose in the blood plasma. This typically refers to a blood glucose reading of higher than 200 mg/dL. Diabetes is a group of metabolic disorders in which the patient has hyperglycemia over a prolonged period of time. Severe complications can arise during prolonged or extreme hyperglycemia. Diabetes is due to the pancreas not producing enough insulin, or the cells of the body not properly responding to insulin. Hypoglycemia is the converse medical condition in which the patient has too little glucose in the blood plasma which can be buffered by physiologic changes, but in severe cases can result in coma, seizures, and even death.

Continuous Glucose Monitors (CGMs) are small sensors that are inserted under the skin that periodically measure interstitial glucose levels. CGMs can record data and store it, as well as produce alarms if dangerous interstitial glucose levels are detected. Typically, CGMs are used in conjunction with fingerstick blood tests to guide patient treatment decisions.

Brain activity refers to action potentials occurring in the brain. Brain activity can be recorded using a wide array of different measurement techniques which all produce different views on brain activity. For example, electroencephalograms (EEGs) use electrode sensors on the scalp to record electrical signals in the brain in a lower resolution fashion compared to brain activity recorded via an electrocorticogram (ECOG) which uses electrode sensors implanted into or on the surface of the brain. Further still, electrodes implanted into the brain may have even higher spatial resolution of the thousands and tens of thousands of neurons (local field potentials) or order of single or small groups of neurons (so called single or multi-unit records). Microelectrode arrays or single electrodes can be implanted into the brain structures to record individual action potentials (or “spikes”) in a particular region of interest.

### SUMMARY OF THE INVENTION

Systems and methods for predictive glucose in accordance with embodiments of the invention are illustrated. One embodiment includes glucose management device, including a brain signal recorder, and a controller, including a processor, and a memory, the memory containing a glucose monitoring application configured to direct the processor to record a brain activity signal of a user's brain using the brain signal recorder, and decode the brain activity signal to predict future glucose levels of the patient.

In another embodiment, the glucose monitoring application further directs the processor to provide a warning when estimated glucose levels rise above a threshold value.

In a further embodiment, the brain signal recorder is: an electroencephalography (EEG) device; a functional near-infrared spectroscopy (fNIRS) device; an electrocorticography (ECOG) device; a deep-brain stimulation device; and a magnetoencephalography (MEG) device.

In still another embodiment, to decode the brain activity signal, the glucose monitoring application further directs the processor to provide the brain activity signal to a multivariate decoder trained on spectral profiles of intracranial activity.

In a still further embodiment, the brain activity signal describes the spectral profile of the broadband brain activity.

In yet another embodiment, the glucose monitoring application further directs the processor to deliver a therapy to the user based on predicted future glucose levels in order to manage glucose levels in a desired therapeutic range.

In a yet further embodiment, the therapy is insulin provided via an insulin pump to the user.

In another additional embodiment, the therapy is brain stimulation.

In a further additional embodiment, the glucose monitoring application further directs the processor to store the predicted future glucose level in the memory, and validate the predicted future glucose level based on a measured glucose level received from a glucose monitor.

In another embodiment again, the glucose level is selected from the group consisting of: blood glucose level; and interstitial glucose level.

In a further embodiment again, A method of managing glucose levels, including recording a brain activity signal of a user's brain using a brain signal recorder, and decoding the brain activity signal to predict future glucose levels of the patient.

In still yet another embodiment, the method further includes providing a warning when estimated glucose levels rise above a threshold value.

In a still yet further embodiment, the brain signal recorder is selected from the group consisting of: an electroencephalography (EEG) device; a functional near-infrared spectroscopy (fNIRS) device; an electrocorticography (ECOG) device; a deep-brain stimulation device; and a magnetoencephalography (MEG) device.

In still another additional embodiment, the method further includes decoding the brain activity signal comprises providing the brain activity signal to a multivariate decoder trained on spectral profiles of intracranial activity.

In a still further additional embodiment, the brain activity signal describes the spectral profile of the broadband brain activity.

In still another embodiment again, the method further includes delivering a therapy to the user based on predicted future glucose levels in order to manage glucose levels in a desired therapeutic range.

In a still further embodiment again, the therapy is insulin provided via an insulin pump to the user.

In yet another additional embodiment, the therapy is brain stimulation.

In a yet further additional embodiment, the method further includes storing the predicted future glucose level in the memory, and validating the predicted future glucose level based on a measured glucose level received from a glucose monitor.

In yet another embodiment again, the glucose level is selected from the group consisting of: blood glucose level; and interstitial glucose level.

In still yet another embodiment again, a system for predictively managing glucose levels including a brain activity recorder, a glucose monitor, an insulin pump, and a controller, where the controller is configured to record brain activity of a user using the brain activity recorder, predict a future hyperglycemic state of the patient based on the recorded brain activity, and direct the insulin pump to deliver insulin to avoid the predicted hyperglycemic state.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates a predictive glucose management system in accordance with an embodiment of the invention.

FIG. 2 illustrates a block diagram for a controller in accordance with an embodiment of the invention.

FIG. 3 is a flow chart for a process for managing glucose levels in accordance with an embodiment of the invention.

FIG. 4 is a flow chart for a process for predicting glucose levels in accordance with an embodiment of the invention.

### DETAILED DESCRIPTION

Metabolic syndromes and diabetes are increasingly prevalent health conditions, now affecting a broader range of ages in our global population. Specifically, the significant morbidity and mortality associated with diabetes have created a major toll on the healthcare system, including extensive costs, both personal and societal, in the form of medical expenditures for the disease itself and loss of workforce productivity from disability associated with disease progression. Hence, the ability to prevent development of diabetes and its subsequent complications is a high-impact area of public health for intervention. Additionally, eating behavior and physiologic regulation of the body's metabolic and weight balance entails a complex interplay of hormonal signaling and behaviors. In this context, close monitoring and control of blood glucose levels has been shown to be one of the best and most reliable methods to prevent complications of both hypo- and hyperglycemia.

Controlling blood glucose is important beyond the scope of diabetes, as both hyper- and hypoglycemia in hospitalized and critically-ill patients are associated with increased cost, length of stay, morbidity, and morality. Patients, especially those in intensive care units may suffer from stress-related hyperglycemia as a result of severe injuries and illnesses, e.g. traumatic brain injury (TBI), intracranial hemorrhage, stroke, subarachnoid hemorrhage (SAH), and many others. Conservative glycemic control has been associated with better outcomes in these patients.

Current iterations of continuous glucose monitors (CGMs) rely on interstitial glucose measurements as a proxy for blood glucose levels, which have an intrinsic lag time and are subject to interference by medications and extreme blood glucose values. Conventional CGMs are also unable to anticipate abnormal glucose levels; as a reactive modality, they can only respond to hypo or hyperglycemia once the abnormality has already occurred. In addition, patients with severe disease may have chronically elevated glucose levels that are refractory to conventional treatment options including continuous insulin infusion. Systems and methods described herein seek to rectify these limitations by predicting what a patient's glucose levels will be in the next several hours. Using this information, preemptive treatment can be delivered to the patient in order to avoid deleterious glucose levels from occurring.

In many embodiments, predictive glucose management (PGM) systems and methods described herein decode brain activity in order to predict the patient's future glucose levels. In a variety of embodiments, brain activity is measured using a non-invasive modality such as electroencephalography (EEG), functional near-infrared spectroscopy (fNIRS), magnetoencephalography (MEG), or any other modality as appropriate to the requirements of specific applications of embodiments of the invention. However, brain activity can be recorded using intracranial sensors if available, such as (but not limited to) deep brain stimulation (DBS) systems, or ECOG. In various embodiments, PGM systems are wearable or otherwise minimally invasive with respect to a patient's life outside of a clinical setting.

In numerous embodiments, systems and methods described herein involve closed-loop management of glucose levels, where preemptive treatment is provided to the patient to avoid hyper- or hypo-glycemia. For example, patients may be delivered long-acting insulin, an insulin analogue, and/or any other hyperglycemia control drug as appropriate to the requirements of specific applications of embodiments of the invention in anticipation of future glucose changes. By way of further example, brain stimulation may be provided in order to perturb the glucose encoding network in the brain in order to alter blood glucose levels for the subsequent several hours. Brain stimulation may be provided by an already implanted DBS electrode depending on implantation location, any other type of implanted electrode, or via a non-invasive brain stimulation modality such as (but not limited to) transcranial magnetic stimulation (TMS), transcranial direct current stimulation (tDCS), transcranial focused ultrasound (tFUS), and/or any other modality as appropriate to the requirements of specific applications of embodiments of the invention. In various embodiments, brain stimulation modalities may be utilized as an adjunct to insulin when the patient is refractory to standard treatments. PGM system architectures are discussed in further detail below.

### Predictive Glucose Management Systems

PGM systems record and decode brain activity to estimate likely glucose levels of a patient in the next several hours. Typically, the predictions are accurate out for at least 2-8 hours, although depending on the patient and condition, this number may increase. In many embodiments, PGM systems provide these predictions to the patient and/or medical professionals. However, in various embodiments, PGM systems are further capable of closed-loop glucose level control by continuously predicting future glucose levels and altering treatment (e.g. drug delivery rate, brain stimulation, etc.) to avoid the predicted deleterious change in glucose level. In this way, PGMs can perform as an artificial pancreas system with superior glucose management capabilities. In some embodiments, patient and/or medical professional authorization is required before treatment is delivered and/or modified by the PGM.

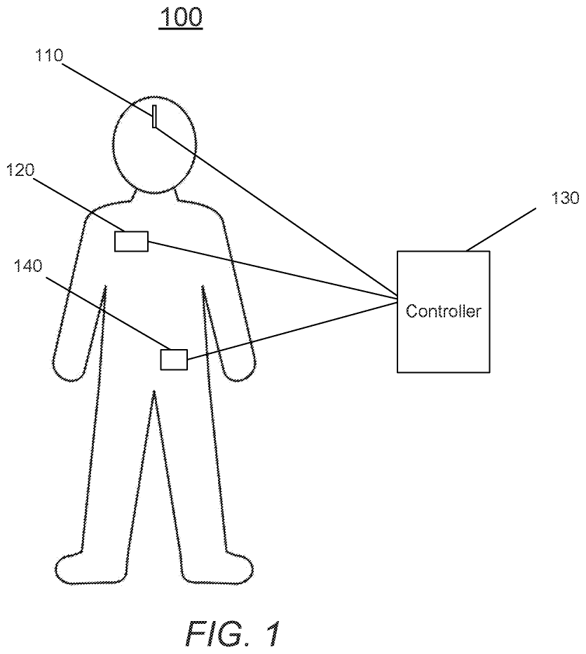

Turning now to FIG. 1, an example PGM system architecture in accordance with an embodiment of the invention is illustrated. PGM system 100 includes a brain activity recorder 110. In the illustrated embodiment, brain activity recorder 110 is a deep brain stimulation system. However, as can readily be appreciated, any brain activity recorder can be used, including those that are non-invasive as discussed above. In some embodiments, the brain activity recorder is a wearable device, rather than an implanted device. PGM system 100 further includes a CGM 120. In many embodiments CGMs are used to continuously confirm the accuracy interstitial blood glucose predictions, and may further act as a redundant warning modality. However, CGMs may not be present in all PGM systems as appropriate to the requirements of specific applications of embodiments of the invention.

A controller 130 is communicatively coupled with the brain activity recorder 110, the CGM 120, and an insulin infusion pump 140. In many embodiments, the communication between different components may not be direct. For example, a brain activity recorder may provide data to a CGM which in turn is provided to the controller rather than communicating with the controller directly. Indeed, as one of ordinary skill in the art would appreciate, any communications architecture can be used without departing from the scope or spirit of the invention.

In numerous embodiments, controllers process recorded brain activity to generate predictions regarding the patient's glucose levels. Controllers may provide predictions for only one of interstitial or blood glucose levels. In some embodiments both interstitial and blood glucose level predictions are computed. Further, controllers may be implemented using any of a variety of computing platforms. In various embodiments, the controller is a smart phone, a smart watch, a tablet computer, a personal computer, and/or any other personal wearable device. In some embodiments, the controller may be integrated into a medical device or a medical server system, e.g. a hospital computer network, or cloud medical system.

In various embodiments, insulin infusion pumps can variably infuse insulin as dictated by the controller. Further, other drugs rather than insulin may be provided via a similar infusion pump depending on the needs of the patient. As can be readily appreciated, many PGM systems may not include any infusion pumps if drug delivery is unadvisable for the particular patient. Similarly, PGMs may further include methods for delivering brain stimulation as an alternative treatment. In various embodiments, the brain activity recorder may also function as a brain stimulation device. Indeed, any number of different PGM system architectures can be used depending on the needs of a specific patient as appropriate to the requirements of specific applications of embodiments of the invention.

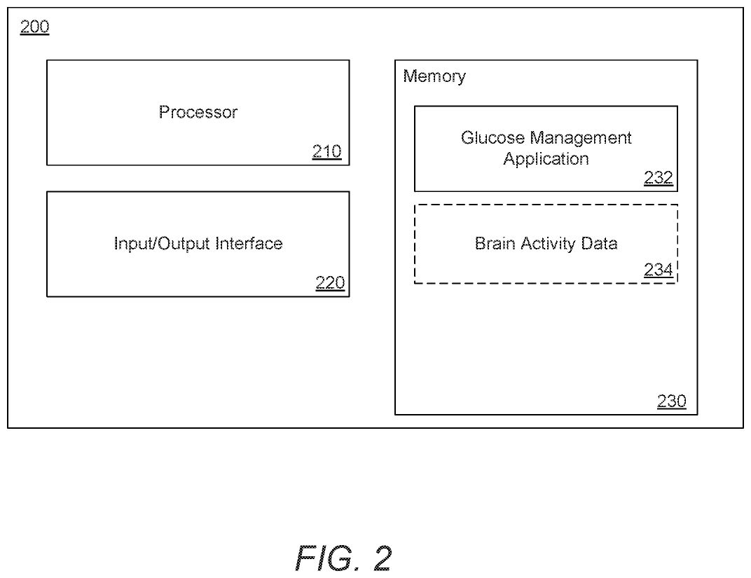

Turning now to FIG. 2, a block diagram for a controller in accordance with an embodiment of the invention is illustrated. Controller 200 includes a processor 210. In many embodiments, the processor is a logic circuit capable of executing instructions such as (but not limited to) a central processing unit (CPU), a graphics processing unit (GPU), a field programmable gate array (FPGA), an application-specific integrated circuit (ASIC), and/or any combination thereof. In many embodiments, more than one processor can be used. The controller 200 further includes an input/output (I/O) interface 220. The I/O interface can be used to communicate with different PGM system components and/or 3^{rd} party components such as (but not limited to) displays, speakers, CGMs, brain activity recorders, stimulation devices, infusion pumps, cellphones, medical devices, computers, and/or any other component via wired or wireless connections.

The controller 200 further includes a memory 230. The memory 230 can be made of volatile memory, nonvolatile memory, or any combination thereof. The memory 230 contains a glucose management application 232. The glucose management application can direct the processor to carry out various PGM processes as described herein. In many embodiments, the memory 230 further contains brain activity data obtained from brain activity recorders. Brain activity data can describe brain activity as a signal or set of signals. In some embodiments, brain activity data includes waveforms recorded by sensor electrodes. In various embodiments, one or more waveforms are recorded for each electrode (“channel”). In a number of embodiments, the brain activity data describes the spectral profile of broadband brain activity. In a variety of embodiments, the glucose management application configures the processor to act as a multivariate decoder for brain activity data. As can be readily appreciated, controllers can be manufactured in different ways using similar computing components without departing from the scope or spirit of the invention. PGM processes are discussed in further detail below.

### Predictive Glucose Management

PGM processes involve the collection and use of brain activity to predict future glucose levels of a patient. In numerous embodiments, treatment recommendations or the treatments themselves can be triggered by a prediction of hyper- or hypoglycemia in order to stabilize the glucose levels at a healthier range. Peripheral glucose levels tend to largely follow circadian dynamics and are strongly coherent to intracranial high frequency activity (HFA, 70-170 Hz) across multiple brain regions. As such, whole brain activity can be used in the predictive modeling process. In some embodiments, brain activity data from known glucose-sensors such as the hypothalamus, amygdala, and hippocampus are used instead of or in conjunction with brain activity from other regions and/or the whole brain.

In order to process data coming from one or more brain activity recorders, a machine learning model can be trained. In some embodiments, the training process is performed using data acquired from the patient on which the trained model will be used. In various embodiments, the model can be pre-trained on standardized data and training can be completed using patient data. In various embodiments, the model is continuously refined using predictions and subsequent validation as measured using a CGM. While linear models are often considered to be less predictive than more modern machine learning models, in many embodiments a linear model is sufficient for accurate prediction. However, in various embodiments, more complex predictive machine learning models can be used, such as (but not limited to) other types of regression models, neural networks, and others as appropriate to the requirements of specific applications of embodiments of the invention.

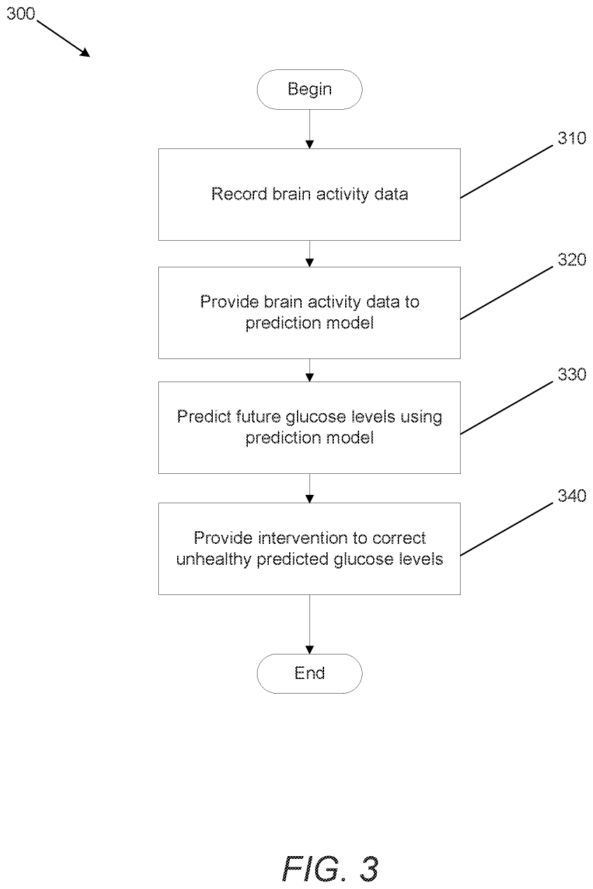

Turning now to FIG. 3, a flow chart for a PGM process for predictively managing glucose levels in accordance with an embodiment of the invention is illustrated. Process 300 includes recording (310) brain activity using a brain activity recorder, and providing (320) the brain activity data to a trained prediction model. The prediction model predicts (330) future glucose levels. In many embodiments, the certainty of the prediction may decrease the further in the future the prediction is made. In various embodiments, multiple predictions are provided at different time points, and only those above a predetermined confidence threshold as determined by a medical professional are used. In some embodiments, a hard limit is set on how far ahead the predictions are made for. If a hypo- and/or hyperglycemic state is predicted, a medical intervention is provided (340) to avoid the unhealthy dip or spike, respectively, in glucose levels. In various embodiments, the medical intervention is automatically provided, e.g. via control of an infusion pump and/or via brain stimulation. In various embodiments, a warning is provided to the patient and/or medical professional that an unhealthy glucose level is predicted. In some embodiments, confirmation is required before the medical intervention is provided.

While a particular process is illustrated in FIG. 3, as can readily be appreciated, various modifications can be made without departing from the scope or spirit of the invention. For example, pre-recorded brain activity data can be provided and used to make predictions. Further, interventions need not be recommended nor provided in all cases. In many situations, it is beneficial to merely have a warning.

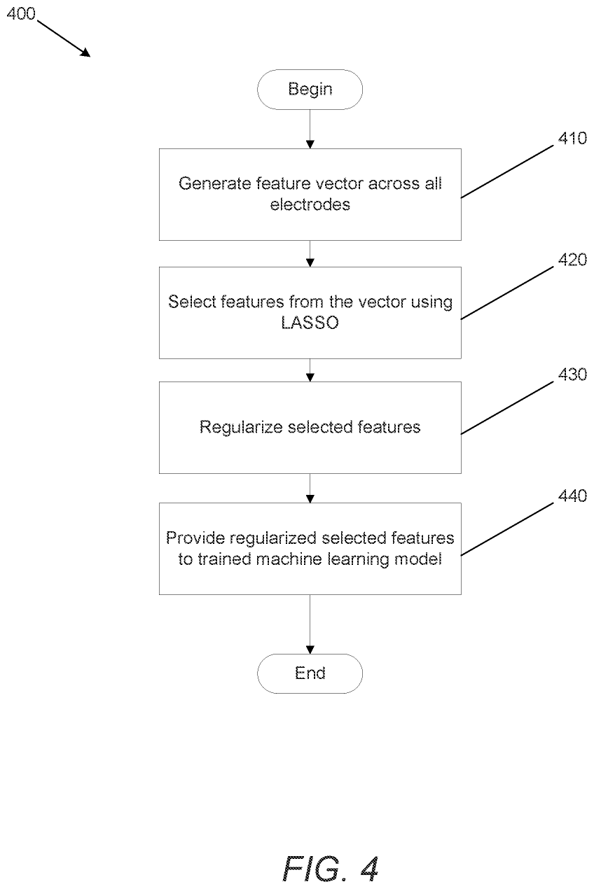

Turning now to FIG. 4, a flow chart of a PGM process for predicting glucose levels based on brain activity data in accordance with an embodiment of the invention is illustrated. Process 400 includes generating (410) a feature vector across all electrode (or sensor) channels. In numerous embodiments, all frequency bands across all channels are flattened into a single feature vector. A Least absolute shrinkage and selection operator (LASSO) model is used to select (420) a subset of features from the feature vector for regularization (430). The regularized features are provided (440) to the trained machine learning model to produce one or more predictions. In numerous embodiments, a similar process is used to train the model using labeled training data from the patient and/or other patients. While specific machine learning models are discussed herein, many different machine learning models can be used without departing from the scope or spirit of the invention.

Although specific methods of PGM are discussed above, many different methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A glucose management device, comprising:
a brain signal recorder; and
a controller, comprising:
a processor; and
a memory, the memory containing a glucose monitoring application configured to direct the processor to:
record a brain activity signal of a user's brain using the brain signal recorder; and
decode the brain activity signal to predict future glucose levels of the patient.

2. The glucose management device of claim 1, wherein the glucose monitoring application further directs the processor to provide a warning when estimated glucose levels rise above a threshold value.

3. The glucose management device of claim 1, wherein the brain signal recorder is selected from the group consisting of: an electroencephalography (EEG) device; a functional near-infrared spectroscopy (fNIRS) device; an electrocorticography (ECoG) device; a deep-brain stimulation device; and a magnetoencephalography (MEG) device.

4. The glucose management device of claim 1, wherein to decode the brain activity signal, the glucose monitoring application further directs the processor to provide the brain activity signal to a multivariate decoder trained on spectral profiles of intracranial activity.

5. The glucose management device of claim 1, wherein the brain activity signal describes the spectral profile of the broadband brain activity.

6. The glucose management device of claim 1, wherein the glucose monitoring application further directs the processor to deliver a therapy to the user based on predicted future glucose levels in order to manage glucose levels in a desired therapeutic range.

7. The glucose management device of claim 6, wherein the therapy is insulin provided via an insulin pump to the user.

8. The glucose management device of claim 6, wherein the therapy is brain stimulation.

9. The glucose management device of claim 1, wherein the glucose monitoring application further directs the processor to:
store the predicted future glucose level in the memory; and
validate the predicted future glucose level based on a measured glucose level received from a glucose monitor.

10. The glucose management device of claim 1, wherein the glucose level is selected from the group consisting of: blood glucose level; and interstitial glucose level.

11. A method of managing glucose levels, comprising:
recording a brain activity signal of a user's brain using a brain signal recorder; and
decoding the brain activity signal to predict future glucose levels of the patient.

12. The method of managing glucose levels of claim 11, further comprising providing a warning when estimated glucose levels rise above a threshold value.

13. The method of managing glucose levels of claim 11, wherein the brain signal recorder is selected from the group consisting of: an electroencephalography (EEG) device; a functional near-infrared spectroscopy (fNIRS) device; an electrocorticography (ECoG) device; a deep-brain stimulation device; and a magnetoencephalography (MEG) device.

14. The method of managing glucose levels of claim 11, decoding the brain activity signal comprises providing the brain activity signal to a multivariate decoder trained on spectral profiles of intracranial activity.

15. The method of managing glucose levels of claim 11, wherein the brain activity signal describes the spectral profile of the broadband brain activity.

16. The method of managing glucose levels of claim 11, further comprising delivering a therapy to the user based on predicted future glucose levels in order to manage glucose levels in a desired therapeutic range.

17. The method of managing glucose levels of claim 16, wherein the therapy is insulin provided via an insulin pump to the user.

18. The method of managing glucose levels of claim 16, wherein the therapy is brain stimulation.

19. The method of managing glucose levels of claim 11, further comprising:
storing the predicted future glucose level in the memory; and
validating the predicted future glucose level based on a measured glucose level received from a glucose monitor.

20. The method of managing glucose levels of claim 11, wherein the glucose level is selected from the group consisting of: blood glucose level; and interstitial glucose level.
